(19) United States
(12) Patent Application Publication
(10) Pub. No.: US 2019/0151369 A1
(43) Pub. Date: May 23, 2019

(54) Engineered Stem Cell Therapy for Cardiac Repair

(71) Applicants: Emory University, Atlanta, GA (US); The UAB Research Foundation, Birmingham, AL (US)

(72) Inventors: Young-sup Yoon, Atlanta, GA (US); Kiwon Ban, Gyeonggi-do (CN); Ho-Wook Jun, Birmingham, AL (US)

(21) Appl. No.: 16/266,975

(22) Filed: Feb. 4, 2019

Related U.S. Application Data
(63) Continuation of application No. 14/716,988, filed on May 20, 2015.
(60) Provisional application No. 62/000,711, filed on May 20, 2014.

(51) Int. Cl. A61K 35/34; C12N 5/077; A61K 38/10

(52) U.S. Cl. CPC A61K 35/34; C12N 2506/02; A61K 38/10; C12N 5/0657

(57) ABSTRACT

This disclosure relates to compositions and methods of cardiac repair. In certain embodiments, the disclosure relates to a composition comprised of peptide amphiphiles and cardiac and/or cardiac precursor cells, and administering such a composition to a subject for the treatment of a cardiac condition. In certain embodiments, the peptide amphiphiles comprise a cell adhesive sequence and a metalloprotease degradable sequence.

### CROSS-REFERENCE TO RELATED APPLICATIONS

This application is a continuation of U.S. application Ser. No. 14/716,988 filed May 20, 2015, which claims the benefit of U.S. Provisional Application No. 62/000,711 filed May 20, 2014. The entirety of each of these applications is hereby incorporated by reference for all purposes.

### STATEMENT REGARDING FEDERALLY FUNDED RESEARCH

This invention was made with government support under HHSN268201000043C and CBET-0939511 awarded by the National Institutes of Health and the National Science Foundation. The government has certain rights in the invention.

### INCORPORATION-BY-REFERENCE OF MATERIAL SUBMITTED AS A TEXT FILE VIA THE OFFICE ELECTRONIC FILING SYSTEM (EFS-WEB)

The Sequence Listing associated with this application is provided in text format in lieu of a paper copy, and is hereby incorporated by reference into the specification. The name of the text file containing the Sequence Listing is 14034USCON_ST25.txt. The text file is 4 KB, was created on Feb. 4, 2019, and is being submitted electronically via EFS-Web.

### BACKGROUND

Heart disease is a leading cause of death in the United States. The majority of fatalities are due to coronary artery disease and correlating heart failure. (Roger et al., 2012, Circulation, 125(1):e2-e220). Although the only therapeutic option for end-stage heart failure is full heart transplantation, the number of donor hearts available and the difficulties in matching patients to donors severely limits the success and viability of full organ transplantation. (Bu et al., 2009, Nature, 460(7251):113-117). Therefore, therapeutic alternatives are needed.

Cardiomyocytes (CMs) derived from pluripotent stem cells (PSCs) including both embryonic stem cells (ESCs) and induced pluripotent stem cells (iPSCs) (Takahashi et al., 2007, Cell, 131(5):861-872; Park et al., 2008, Cell, 134(5):877-886) are a source of cells for cardiac repair (Zwi et al., 2009, Circulation, 120(15):1513-1523). CMs differentiated from both of these PSC types have cardiomyogenic potential. (Kattman et al., 2011, Cell Stem Cell, 8(2):228-240) (Burridge et al., 2012, Cell Stem Cell, 10(1):16-28). Moreover, PSC-derived CMs exhibit spontaneous contractile activity and cardiac-type mechanisms of excitation-contraction coupling, and expression of expected sarcomeric proteins, ion channels, and transcription factors (Mignone et al., 2010, Circ J, 74(12):2517-2526). Several recent pre-clinical studies have shown that transplantation of PSC derived CMs into rodent infarct models helped preserve cardiac function. (Yuasa et al., 2005, Nat Biotechnol, 23(5):607-611; Nemir et al., 2006, Circ Res, 98(12):1471-1478; Mummery et al., 2003, Circulation, 107(21):2733-2740; Passier et al., 2005, Stem Cells, 23 (6):772-780).

Engraftment of delivered CMs into heart is low and their survival rate is poor. Particularly, direct injection of cells alone is hampered by survival of only 50% of cells immediately after injection, and only 10% survival of the remaining cells after 1 week. (Müller-Ehmsen et al., 2002, J Mol Cell Cardiol, 34(2):107-116; Zhang et al., 2001, J Mol Cell Cardiol, 33(5):907-921). The longest reported survival of hPSC-CM after direct injection into murine myocardium is 12 weeks. (Van Laake et al., 2008, Circ Res, 102(9):1008-1010). Thus, there is a need to develop methods to support retaining the cells in a localized manner and to promote cell viability, differentiation, maturation and integration with the host myocardium by providing the cells with a nurturing environment that protects and regulates the behaviors of the cells. (Itescu, et al., 2003, J Mol Med (Berl), 81(5):288-296; Dowell et al., 2003, Cardiovasc Res, 58(2):336-350; Rubart et al., 2004, J Clin Invest, 114(6):775-783).

Anderson et al. report osteogenic differentiation of human mesenchymal stem cells directed by extracellular matrix-mimicking ligands in a biomimetic self-assembled peptide amphiphile nanomatrix (2009, Biomacromolecules, 10(10):2935-2944).

Kim et al. report self-assembling peptide amphiphile-based nanofiber gel for bioresponsive cisplatin delivery (2009, Mol Pharm 6(3): 978-985

Dawson et al. report collagen scaffolds with or without the addition of RGD peptides support cardiomyogenesis after aggregation of mouse embryonic stem cells. (2011, In Vitro Cell Dev Biol Anim, 47(9):653-64).

Mayfield et al. report the effect of encapsulation of cardiac stem cells within matrix enriched hydrogel capsules on cell survival, post-ischemic cell retention and cardiac function (2014, Biomaterials, 35(1): 133-142).

Tongers et al. report RGDS-epitope presenting peptide amphiphile nanofibers enhance regenerative potency of cell-based strategies in ischemic tissue (2008, Circulation, 118:S509).

Stupp et al. reports methods and materials for nanocrystalline surface coatings and attachment of peptide amphiphile nanofibers thereon. U.S. Pat. No. 7,390,526.

Webber et al. reports development of bioactive peptide amphiphiles for therapeutic cell delivery (2010, Acta Biomater, 6(1): 3-11).

Jun et al. report technologies for pancreatic islet transplantation (WO2013/3134360). See also Hartgerink et al., 2001, Science, 294(5547):1684-1688; Hartgerink et al., 2002, Proc Natl Acad USA; 99(8):5133-5138 (Beniash et al., 2005, Acta Biomater, 1(4): 387-397; Jun et al., 2006, Soft Matter, 2:177-181.

U.S. Pat. No. 8,735,354 reports an endothelium mimicking nanomatrix.

References cited herein are not an admission of prior art.

### SUMMARY

This disclosure relates to compositions and methods of treating or preventing a cardiovascular disease or condition. In certain embodiments, the disclosure relates to a composition comprised of peptide amphiphiles and cardiac and/or cardiac precursor cells, and administering such a composition to a subject for the treatment of a cardiovascular disease or condition. In certain embodiments, the peptide amphiphiles comprise a cell adhesive sequence and a metalloprotease degradable sequence.

In certain embodiments, the disclosure relates to compositions comprising a) a peptide amphiphile comprising a cell adhesive peptide sequence and a protease degradable sequence linked to a hydrocarbon; and b) a cardiac cell or a cardiac precursor cell. In certain embodiments, the cardiac cell or cardiac precursor cell is made by the process of culturing isolated cells, e.g., stem cells or induced pluripotent cells, and a cardiac inducible molecule under conditions such that culturing/replication results in pacemaker-like or primary myocardial-like cells, intermediate cardiac cells, or atrial-, ventricular-, nodal, His-, and Purkinje-like cells.

In certain embodiments, cell culturing/replication results in a cardiac cell or a cardiac precursor cell that spontaneously contract.

In certain embodiments, the isolated cell is an embryonic stem (ES) cell or embryoid body cell.

In certain embodiments, the cardiac inducible molecule is selected from ascorbic acid, icariin, icaritin, desmethylicaritin, bone morphogenetic protein (BMP)-2 or BMP-4, dimethyl sulfoxide.

In certain embodiments, cell replication results in cells that that express a cardiac-specific nucleic acid selected from Nkx2.5, α-actin, MLC-2v, and MLC-2a.

In certain embodiments, the isolated cell is isolated from a sample by fluorescence-activated cell sorting after mixing with a molecular beacon targeting a cardiac-specific nucleic acid.

In certain embodiments, the isolated cell is isolated from bone marrow or peripheral blood, by selecting cells that express CD31 and/or CD34.

In certain embodiments, the peptide amphiphile is alkyl-CONH-GTAGLIGQ-RGDS (SEQ ID NO: 1) or variants thereof.

In certain embodiments, the peptide amphiphile is alkyl-CONH-GTAGLIG (SEQ ID NO: 2)-poly-lysine, e.g., alkyl-CONH-GTAGLIG-KKKK (SEQ ID NO: 18), alkyl-CONH-GTAGLIG-KKKKK (SEQ ID NO: 19), or alkyl-CONH-GTAGLIG-KKKKKK(SEQ ID NO: 20).

In certain embodiments, the compositions comprise the amphiphile peptides as a combination of is alkyl-CONH-GTAGLIGQ-RGDS(SEQ ID NO: 1) and alkyl-CONH-GTAGLIG(SEQ ID NO: 2)-poly-lysine.

In certain embodiments, alkyl is CH₃(CH₂)ₙ—, e.g., CH₃(CH₂)₁₄—, wherein n is 6 or more, e.g., 6-20.

In certain embodiments, compositions comprising a) a peptide amphiphile comprising a cell adhesive peptide sequence and a protease degradable sequence linked to a hydrocarbon also contains a peptide amphiphile without a cell adhesive peptide sequence. In certain embodiments, the ratio of peptide amphiphiles containing a cell adhesive sequence to peptide amphiphiles without a cell adhesive sequence is between 1:1 to 1:2, or 1:2 to 1:3, or 1:3 to 1:4, or 1:4 to 1:5, or 1:5 to 1:10, or 2:1 to 1:1, or 3:1 to 2:1, or 4:1 to 3:1, or 5:1 to 4:1, or 10:1 to 5:1.

In certain embodiments, the disclosure relates to methods of treating or preventing cardiac condition comprising administering a composition disclosed herein, e.g., comprising cultured cardiac cells or cardiac precursor cells and a peptide amphiphile disclosed herein in an effective amount to a subject in need thereof. In certain embodiments, the cardiac condition is peripheral vascular disease, myocardial ischemia, heart failure, or stroke. In certain embodiments, the subject is a human.

In certain embodiments, the disclosure relates to a composition comprising a peptide comprising a cell adhesive sequence and a second sequence, wherein the second sequence comprise between 5 and 20 amino acids wherein more than 50 percent of the amino acids are glycine or a hydrophilic amino acid; wherein the second sequence is linked to a hydrocarbon with a carbon chain of greater than 6; and a cardiac cell or a cardiac precursor cell.

In certain embodiments, the disclosure relates to compositions comprising a cell adhesive sequence that is an integrin binding cell adhesive ligand. In certain embodiments, the cell adhesive sequence is RGD (SEQ ID NO: 16). In certain embodiments, the second sequence is GTAGLIGQ (SEQ ID NO: 2). In certain embodiments, the hydrocarbon contains an 8 to 22 carbon chain.

In certain embodiments, the composition includes any human cell such as a cardiac precursor cell that is a pluripotent stem cell. In certain embodiments, the composition includes a pluripotent stem cell which is an embryonic stem cell. In certain embodiments, the composition includes a human pluripotent stem cell which is a human embryonic induced pluripotent stem cell. In certain embodiments, the composition includes a cardiac precursor cell that is isolated from bone marrow or peripheral blood, by selecting cells that express CD31. In certain embodiments, the gel composition includes a cardiac precursor cell that is an endothelial progenitor cell.

In certain embodiments, the composition includes a cardiac cell which is a cardiomyocyte. In certain embodiments, the composition includes a cardiomyocyte which is derived from a pluripotent stem cell. In certain embodiments the composition includes a cardiomyocyte which is derived from an embryonic stem cell. In certain embodiments the composition includes a cardiomyocyte which is derived from an induced pluripotent stem cell. In certain embodiments, the composition includes a cardiomyocyte which is a ventricular cardiomyocyte. In certain embodiments, the composition includes a ventricular cardiomyocyte which is isolated using a molecular beacon technology.

In certain embodiments, for any of the compositions or uses disclosed herein the cells are selected from human induced pluripotent stem cell or embryonic stem cell-derived cardiomyocytes, endothelial cells and lymphatic endothelial cells and bone marrow-derived CD31+cells and mesenchymal stem cells.

In certain embodiments, the disclosure relates to a method of treating or preventing a cardiac condition in a patient comprising, combining cultured cardiac cells or cardiac precursor cells with a peptide comprising a cell adhesive sequence and a second sequence, wherein the second sequence comprise between 5 and 20 amino acids wherein more than 50 percent of the amino acids are glycine or a hydrophilic amino acid; wherein the second sequence is linked to a hydrocarbon with a carbon chain of greater than 6; and administering said mixture in an effective amount to a patient in need thereof. In certain embodiments, the disclosure relates to methods of treating or preventing cardiac condition wherein the vascular disease or condition is peripheral vascular disease, myocardial ischemia, heart failure, or stroke.

In certain embodiments, the disclosure relates to a method of growing cardiac cells or cardiac precursor cells in culture comprising culturing cardiac cells or cardiac precursor cells with a peptide comprising a cell adhesive sequence and a second sequence, wherein the second sequence comprise between 5 and 20 amino acids wherein more than 50 percent of the amino acids are glycine or a hydrophilic amino acid; and wherein the second sequence is linked to a hydrocarbon with a carbon chain of greater than 6. In certain embodiments, the disclosure relates to a method of growing cardiac cells or cardiac precursor cells in culture, wherein said culture is used for screening of drugs for potential treatment of a cardiac condition. In certain embodiments, the disclosure relates to a method of growing cardiac cells or cardiac precursor cells in culture, wherein said culture is used for screening of drugs for potential treatment of cardiac condition, wherein the cardiac condition is peripheral vascular disease, myocardial ischemia, heart failure, or stroke.

In certain embodiments, the disclosure relates to a method of enhancing stem cell therapy by promoting cell viability, differentiation, maturation and integration with host myocardium comprising combining cultured cardiac cells or cardiac precursor cells with a peptide comprising a cell adhesive sequence and a second sequence, wherein the second sequence comprise between 5 and 20 amino acids wherein more than 50 percent of the amino acids are glycine or a hydrophilic amino acid; wherein the second sequence is linked to a hydrocarbon with a carbon chain of greater than 6; and administering said mixture in an effective amount to a patient in need thereof.

### BRIEF DESCRIPTION OF THE DRAWINGS

FIG. 1 schematically illustrates embodiments of the disclosure wherein PA-RGDS is [CH₃(CH₂)₁₄CONH-GTAGLIGQ-RGDS] (SEQ ID NO: 1).

FIG. 2 shows data on the evaluation of cellular behaviors of cardiomyocytes encapsulated in PA-RGDS. Representative Live/Dead assay images of NRCMs after 7 days of culture in normoxic conditions are summarized in the bar graph.

FIG. 3A shows data on the cytoprotective effects of PA-RGDS encapsulation against H₂O₂ indicating encapsulation of NRCMs with PA-RGDS increased cell survival after H₂O₂ (200 μM) treatment as determined by the Live/Dead assay.

FIG. 3B shows data on cell viability measured by extracellular release of LDH.

FIG. 4 shows data on quantification of engrafted HL-1 CMs by flow cytometry following cardiac tissue digestion into cell suspension indicating survival and engraftment of HL-1 CMs after injection into uninjured mouse hearts. Seven days after injection of diI-labeled HL-1 CMs encapsulated with or without PA-RGDS into intact mouse hearts, mice were sacrificed and hearts were collected.

FIG. 5A schematically illustrates the generation of highly purified cardiomyocytes from differentiating mESCs. A schematic of the protocol used for differentiating mESCs to the cardiac lineage.

FIG. 5B shows flow cytometry analysis of Tnnt2 expression in the cardiomyogenically differentiated mESC at day 11 and day 18 N=3. The numbers represent the percentages of Tnnt2-positive cells

FIG. 6A shows data indicating favorable effects of mESC-CMs with PA-RGDS on mouse experimental MI. Improvement of cardiac function in mice receiving mESC-derived CMs with PA-RGDS. Fractional shortening (FS: left) and ejection fraction (EF: right) were significantly higher in the mESC-CM+PA-RGDS group compared to the three other groups measured by echocardiography.

FIG. 6B shows quantification results from t four treated groups showing cardiac fibrosis after staining with Masson's trichrome in the hearts harvested 4 weeks after MI.

FIG. 6C shows data on confocal microscopic images of heart sections collected 4 weeks after MI and cell injection indicating that the engraftment of DiI-labeled mESC-CMs was substantially higher when cells were encapsulated.

FIG. 7A shows data indicating sustained therapeutic effects of mESC-CMs with PA-RGDS on a mouse model of MI. EF and FS measured by echocardiography were significantly greater in the mESC-CM+PA-RGDS group compared to the mESC-CM-only injected group.

FIG. 7B shows quantification data of representative confocal microscopic images showing engraftment of DiI-labeled mESC-CMs in hearts harvested at 14 weeks compared to the CM-only injected group and the CMs with PA-RGDS group.

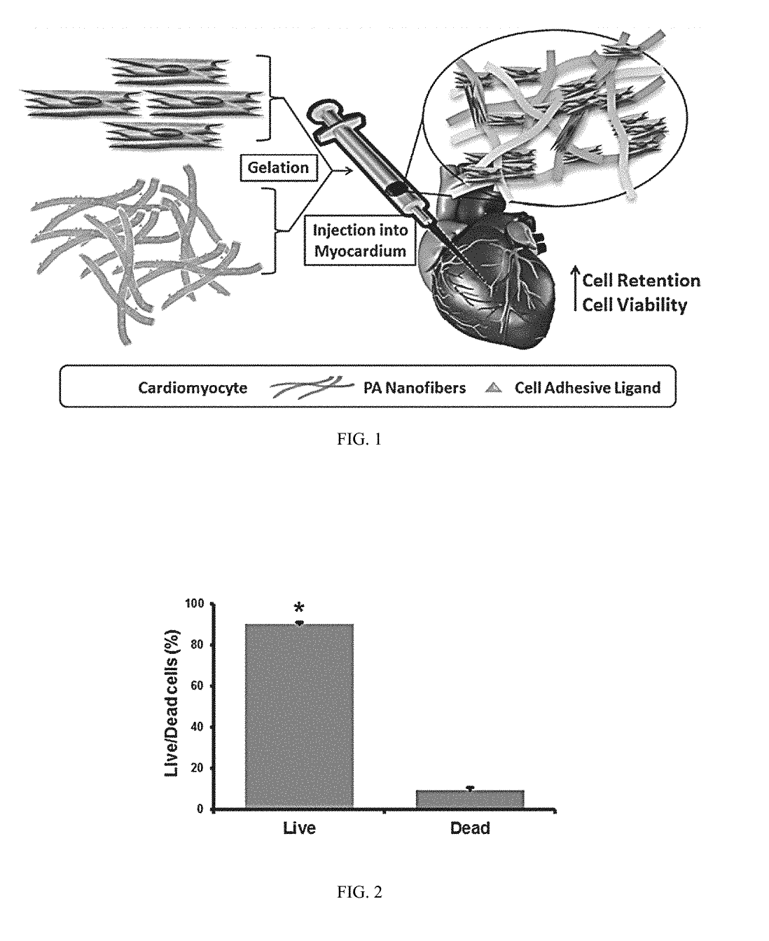

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise.

“Subject” refers any animal, preferably a human patient, livestock, or domestic pet.

The terms “protein” and “polypeptide” refer to compounds comprising amino acids joined via peptide bonds and are used interchangeably. Amino acids may be naturally or non-naturally occurring.

A “variant” refers to a chemically similar sequence because of amino acid changes or chemical derivative thereof. In certain embodiments, a variant contains one, two, or more amino acid deletions or substitutions. In certain embodiments, the substitutions are conserved substitutions. In certain embodiments, a variant contains one, two, or ten or more an amino acid additions. The variant may be substituted with one or more chemical substituents.

One type of conservative amino acid substitutions refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. More rarely, a variant may have “non-conservative” changes (e.g., replacement of a glycine with a tryptophan). Similar minor variations may also include amino acid deletions or insertions (in other words, additions), or both. Guidance in determining which and how many amino acid residues may be substituted, inserted or deleted without abolishing biological activity may be found using computer programs well known in the art, for example, DNAStar software. Variants can be tested in functional assays. Certain variants have less than 10%, and preferably less than 5%, and still more preferably less than 2% changes (whether substitutions, deletions, and so on).

As used herein, the term “derivative” refers to a structurally similar compound that retains sufficient functional attributes of the identified analogue. The derivative may be structurally similar because it is lacking one or more atoms, substituted, multiply substituted, a salt, in different hydration/oxidation states, or because one or more atoms within the molecule are switched, such as, but not limited to, replacing an oxygen atom with a sulfur atom or replacing an amino group with a hydroxyl group and vice-versa. Derivatives may be prepare by any variety of synthetic methods or appropriate adaptations presented in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference. Contemplated substitutions include replacing hydrogen with a substituent such as halogen, hydroxy, carboxy, alkoxy, alkyl, acetyl, amino, alkylamino, dialkylamino, thiol, alkylthiol, phenyl, benzyl, cyclic, heterocyclic, or aromatic group. Substituents may be further substituted or multiply substituted.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g. patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

As used herein, “salts” refer to derivatives of the disclosed compounds where the parent compound is modified making acid or base salts thereof. Examples of salts include, but are not limited to, mineral or organic acid salts of basic residues such as amines, alkylamines, or dialkylamines; alkali or organic salts of acidic residues such as carboxylic acids; and the like. In preferred embodiment the salts are conventional nontoxic pharmaceutically acceptable salts including the quaternary ammonium salts of the parent compound formed, and non-toxic inorganic or organic acids. Preferred salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

### Enhanced Engraftment of Embryonic Stem Cell-Derived Cardiomyocytes Encapsulated in Injectable Nanomatrix Gel

The potential success of stem cell-based therapy for cardiac regeneration has been consistently hindered by a critical inability to retain implanted cells in heart. In most stem cell studies, myocardially injected cells have a very low survival rate. Thus, various natural and artificial biomaterials have been assessed for cardiac repair with adult or pluripotent stem cells.

Cell adhesive ligand Arg-Gly-Asp (RGD) was incorporated into the peptide sequence of a peptide amphiphile (PA), i.e., PA-RGDS is [CH₃(CH₂)₁₄CONH-GTAGLIGQ-RGDS] (SEQ ID NO: 1). Matrix metalloprotease-2 (MMP-2) degradable sequence Gly-Thr-Ala-Gly-Leu-Ile-Gly-Gln (GTAGLIGQ) (SEQ ID NO:2) was also incorporated to provide an enzyme degradable site. RGD is a α1β5 integrin binding cell adhesive ligand found in ECM proteins such as fibronectin and laminin and promotes cell adhesion and viability. A second PA without bioactive cell adhesive ligand, PA-S (C₁₆-GTAGLIGQS)(SEQ ID NO: 17) was also synthesized to provide mechanical strength to the PA gels. Studies were performed to test whether inclusion of RGD sequence, MMP-2 degradable sites, and self-assembly into nanofibers under physiological conditions provide the nanomatrix with ECM mimicking characteristics. Whether the PA-RGDS is safe and biocompatible against CMs and if PA-RGDS can improve the cardiac repair by enhancing structural integration between transplanted mESC-derived CMs and host myocardium was tested.

While the cardiac function of the mESC-CM-only injected group was improved to a similar extent to the mESC-CMs encapsulated with PA-RGDS group by 2 weeks after treatment, suggesting regenerative effects of bare mESC-CMs at an early phase, such effects disappeared after 3 weeks. Histologic examination demonstrated that the mESC-CM-only injected group showed virtually complete disappearance of the injected cells over 14 weeks. However, the PA-RGD S-encapsulated mESC-CM group showed long-term cellular engraftment and integration into host myocardium, expressed mature and structural CM proteins such as cTNT and MHC6/7, formed gap junctions with host CMs, and maintained improved cardiac function, indicating the effectiveness of PA-RGDS on the function of injected mESC-CMs. These results indicate that even though the gels may degrade by week 6, the cells delivered by the gels show significant long-term favorable outcomes. PA-RGDS indeed provides the cells with a protective environment during the early stage of implantation, which is critical for cell engraftment and survival, leading to improved retention and eventual interactions with host tissue.

Cardiomyocytes (CMs) derived from pluripotent stem cells (PSCs) including both embryonic stem cells (ESCs) and the induced pluripotent stem cells (iPSCs) are useful for generating new CMs in injured hearts, with PA-RGDS, which addresses low cell survival. The PA-RGDS provides a protective microenvironment for CMs in vivo. An important aspect of the PA-RGDS is the physical properties of the gels, which can be tuned to provide mechanical strength. The mechanical properties of PA-RGDS self-assembled under conditions determined by the storage modulus (G′) have been observed to be around 100 Pa, which puts them in the category of moderately stiff hydrogels with higher potential for cell encapsulation and delivery for soft tissue engineering applications such as cardiovascular tissues. PA-RGDS nanomatrix gels were also observed to be structurally stable with well-defined cylindrical shape and increased durability for handling and transport. This is particularly important for early cell survival, as cardiac tissue scaffolds require enough mechanical strength to resist the cardiac cycle, but enough flexibility to allow the contractile function of implanted CMs. The mechanical strength of PA-RGDS can be tuned for the application.

### Peptide Amphiphile

The term “peptide amphiphile” as used herein refers to a peptide-based molecule that comprises both a hydrophobic domain and hydrophilic domain. In certain embodiments, the peptide amphiphile has the capability to self-assemble into a three-dimensional network. In certain embodiments, the peptide amphiphiles can self-assemble into a three-dimensional structure that closely mimics the extracellular matrix that surrounds cells in native tissues. In certain embodiments, the peptide amphiphile can provide a protective microenvironment while simultaneously promoting localized extracellular matrix formation and intercellular connections in transplanted cells. In certain embodiments, the peptide amphiphile can promote cardiac regeneration.

In certain embodiments, the hydrophobic domain is comprised of a peptide chain wherein the amino acids that comprise said peptide chain have hydrophobic properties. In certain embodiments, the hydrophobic domain is comprised of a hydrocarbon chain. In certain embodiments, the hydrocarbon chain contains between 2 and 20 carbon molecules. In the preferred embodiment, the hydrocarbon chain contains 16-18 carbon molecules or derivatives.

In certain embodiments, the hydrophilic domain is comprised of a peptide chain wherein the amino acids that comprise said peptide chain have hydrophilic properties. In certain embodiments the hydrophilic peptide chain contains a cell adhesion ligand binding sequence. Examples cell adhesive sequences include, but not limited to, the amino acid sequences DGEA (SEQ ID NO: 3), YIGSR(SEQ ID NO: 4), IKVAV(SEQ ID NO: 5), ERGDS(SEQ ID NO: 6). In certain embodiments, the hydrophilic peptide chain contains an integrin binding cell adhesive ligand sequence. Examples of integrin binding cell adhesive ligand sequences include, but are not limited to, KQAGDV(SEQ ID NO: 7), LDV(SEQ ID NO: 8), IDS (SEQ ID NO: 9), RLD (SEQ ID NO: 10), KRLDS (SEQ ID NO: 11), LET (SEQ ID NO: 12) IET (SEQ ID NO: 13), YYGDLR(SEQ ID NO: 14) FYFDLR (SEQ ID NO: 15) (Ruoslahti et al., 1996, Annu Rev Cell Dev Biol, 12: 697-715). In certain embodiments, the hydrophilic peptide chain contains an α1β5 integrin binding cell adhesive ligand. In certain embodiments, the hydrophilic peptide chain has the amino acid sequence RGD (SEQ ID NO: 16).

In certain embodiments, the peptide amphiphile comprises a second hydrophilic peptide sequence. In certain embodiments the second hydrophilic peptide sequence, is an enzyme mediated degradable site. In certain embodiments, the second hydrophilic peptide sequence is a matrix metalloprotease-2 (MMP-2) degradable sequence. In certain embodiments, the second hydrophilic peptide sequence has the amino acid sequence GTAGLIGQ (SEQ ID NO: 2). In certain embodiments, the second hydrophilic peptide sequence allows progressive degradation of the peptide amphiphile scaffold and replacement by natural extracellular matrix produced by the cells.

Compositions of Cardiac Cells and Cardiac Precursor Cells

Studies have shown that cell cultures directed toward differentiation of pluripotent stem cells into cardiomyocytes include three types of cardiomyocytes: nodal, atrial and ventricular cardiomyocytes. Each type of cardiomyocyte has a unique functional, structural and electrophysiological characteristic, and ventricular cardiomyocytes have been shown desirable for cardiac repair. In certain embodiments, it may be desirable to separate ventricular cardiomyocytes from a general pool of cardiomyocytes which would contain nodal, atrial and ventricular cardiomyocytes.

In certain embodiments, the cardiac precursor cells are human embryonic stem cells which typically express Oct-4, Nanog, Sox2, Nin28, and Dnmt3b. In certain embodiments, the cardiac precursor cells are mesoderm cells which typically express BryT, FoxC1 and Dkk-1. In certain embodiments, the cardiac precursor cells are pre-cardiac mesoderm cells that express Mesp-1, Isl-1, and Flk-1. In certain embodiments, the cardiac precursor cells are cardiac progenitor cells that express Nkx2.5, Tbx5/20, Gata-4, Mef2c, and Hand1/2. In certain embodiments, the cardiac precursor cells are early cardiomyocytes that express cTnT, alpha-actin(c), MHC, MLC2a/v, and Pln.

In certain embodiments, cardiac cells are cells made by the process of producing human ESCs embryoid bodies by a hanging-drop or forced-aggregation, e.g., spinning, allowing for spontaneous differentiation followed by culturing with cardiac inducing growth factors and/or hormones such as activin A, BMP-4, bFGF, Wnt3a, VEGF, DKK1, and combinations thereof.

In certain embodiments, cardiac cells are cells made by the process of co-culturing human ESCs embryoid body cells with cardiomyocytes or division arrested cardiac cells, e.g., human or mouse division arrested END-2 cells. In certain embodiments, the culture medium may contain a P38 MAPK inhibitor and prostaglandin I2 (PG I2).

In certain embodiments, ventricular cardiomyocytes are isolated using molecular beacon based sorting technique. See Ban et al., Circulation, 2013, 128: 1897-1909 and U.S. patent application Ser. No. 14/211,430. Molecular beacon (MB) technology is a method of sorting cells based on mRNA sequences. MBs are 20-30 base pair (bp) oligonucleotide probes with a fluorophore conjugated to the 5′ end and a quencher at the 3′ end. (Heyduk T & Heyduk E, 2002 Nat Biotech, 20:171-176). MBs are designed with 4-7 bps at the 5′ end which are complementary to the bps at the 3′ end. This self-complementary configuration induces the oligonucleotides to form a stem-loop (hairpin) structure so that the fluorophore and the quencher are within close proximity (<7 nm) and fluorescence is quenched. Hybridization of the MBs with the target mRNA opens the hairpin structure and physically separates the fluorophore from the quencher, allowing a fluorescence signal to be emitted upon excitation. (Tsourkas et al., 2002, Nucleic acids research, 30:4208-4215).

MB technology may be used to separate and purify specific cell populations for example specific subpopulations of cardiac cells. In certain embodiments, the molecular beacon targets myosin heavy chain 6/7 mRNA (MHC1-MB). In certain embodiments, MB technology may be used to target transcription factors that specifically identify ventricular cardiomyocytes. In certain embodiments, the MB technology may target the transcription factor Irx4, which is specifically expressed in ventricular cardiomyocytes and not atrial or nodal cardiomyocytes. Other contemplated target cardiac mRNA are selected from myosin heavy chain 1 (MHC1), myosin heavy chain 2 (MHC2), troponin T 1 (TNT1), troponin T 2 (TNT2), troponin T 3 (TNT3). Molecular beacons that target the cardiomyocyte specific mRNA are typically made up of an oligonucleotide sequence of that is complementary to the specific target mRNA. In certain embodiments, the isolated cardiomyocyte population has more than 50% of the cells stain positive for cardiomyocyte markers ACTN2, TNNT2, and TNNI3 by immunohistochemistry.

Cardiac precursor cells include stem cells. They also include precursors of connective tissue cells, nerve cells, and all other types of cells present in the cardiac area. In certain embodiments the cardiac precursor cell may either be a pluripotent stem cell or an adult stem cell.

In certain embodiments the cardiac precursor cell may be a pluripotent stem cell that is an embryonic stem cell. In certain embodiments the cardiac precursor cell may be an induced pluripotent stem cell.

In certain embodiments, the cardiac precursor cell is a multipotent or pluripotent adult stem cell or somatic stem cell. Such stem cells include, but are not limited to, hematopoietic stem cells, bone marrow stromal cells, mesenchymal stem cells, epidermal stem cells and epithelial stem cells.

In certain embodiments, bone marrow cells are a source for cardiac precursor cells. Bone marrow cells have the capacity to repopulate many non-hematopoietic tissues such as vessel and muscle. In certain embodiments, endothelial progenitor cells or mesenchymal stem cells are a source for cardiac precursor cells.

In certain embodiments, cardiac precursor cells are CD31 positive or CD34 positive cells are isolated from a pool of adult stem cells (such as bone marrow cells, peripheral blood). See Kim et al., Blood Res, 2013, 48(2):76-86.

Bone marrow mesenchymal stem cells differentiated into cardiomyocytes after treatment with cardiac inducible molecules such as 5-azacytidine. In certain embodiments, cardiac cells as provided herein are produced by exposure of mesenchymal stem cells or cardiac precursor cells with 5-azacytidine.

In certain embodiments, cardiac precursor cells may be selected from endothelial progenitor cells (EPCs), c-kit positive, sca-1 positive, isl-1 positive, and neural crest stem cells. In certain embodiments, cardiac precursor cells are c-KIT-negative amniotic fluid-derived cells

(ACs) reprogrammed into ECs upon lentiviral expression of a combination of ETV2, ERG, and FLI1 with inhibition of TGFβ signaling.

In certain embodiments, isolated cells as provided herein are purified using a fluorescent dye, tetramethylrhodamine methyl ester perchlorate. Mixing cardiomyocytes with the fluorescent dye labels mitochondria, and the labeled cells can subsequently be enriched by fluorescence-activated cell sorting.

In certain embodiments, the disclosed cardiac or cardiac precursor cells in peptide amphiphile compositions provided herein further comprise fibroblasts.

In certain embodiments, the disclosed cardiac or cardiac precursor cells in peptide amphiphile compositions provided herein further contain components selected from laminin, entactin, collagen I, cyclosporine A, pinacidil, IGF-1, ET-1, EGF, bFGF, PDGF-BB, human fibrin and thrombin.

### Methods of Treatment and Prevention of Cardiac Diseases or Conditions

The pharmaceutical compositions of the present invention may be used as therapeutic agents—i.e. in therapy applications. As herein, the terms “treatment” and “therapy” include curative effects, alleviation effects, and prophylactic effects. In certain embodiments, a therapeutically effective dose of cells is applied, delivered, or administered to the heart or implanted into the heart. An effective dose or amount is an amount sufficient to effect a beneficial or desired clinical result. Said dose could be administered in one or more administrations.

A “cardiac condition” is a state of the heart that can be identified by symptoms or other identifying factors as diverging from a healthy or a normal state. The term “cardiac condition” includes disorders, syndromes, diseases, and injuries that affect the heart. Cardiac conditions include, but are not limited to, cardiac failure, for example, congestive heart failure; ischemic conditions, for example, myocardial infarction; hypertensive conditions; congenital conditions; infectious conditions, for example, endocarditis; and coronary artery disease. Cardiac conditions also include myocardial diseases, for example, myocarditis, cardiomyopathy, and fibrosis; pericardial diseases, for example, pericarditis; and endocardial and valvular diseases, for example, stenosis and prolapse.

In certain embodiments, the disclosure relates to a method of treating or preventing cardiac condition in a patient comprising combining cultured cardiac cells or cardiac precursor cells with a peptide amphiphile disclosed herein, e.g., comprising a cell adhesive sequence and a second sequence, wherein the second sequence comprise between 5 and 20 amino acids wherein more than 50 percent of the amino acids are glycine or a hydrophilic amino acid; wherein the second sequence is linked to a hydrocarbon with a carbon chain of greater than 6; and administering said mixture in an effective amount to a patient in need thereof. Response and effects of the method of treatment include, but are not limited to, increased retention, viability and integration of cardiac stem cells with host tissue; enhanced cardiac repair, decreased scar size, robust engraftment of cardiac stem cells within the myocardium, and expression of representative CM proteins such as cTNT and MIIC6/7 in transplanted cells.

In certain embodiments, the disclosure relates to a method of growing cardiac cells or cardiac precursor cells in culture comprising culturing cardiac cells or cardiac precursor cells with a peptide amphiphile disclosed herein, e.g. comprising a cell adhesive sequence and a second sequence, wherein the second sequence comprise between 5 and 20 amino acids wherein more than 50 percent of the amino acids are glycine or a hydrophilic amino acid; and wherein the second sequence is linked to a hydrocarbon with a carbon chain of greater than 6. In certain embodiments, this method is used for screening of drugs for potential treatment of cardiac condition.

In certain embodiments, the disclosed composition can be used to chest specific effects of cardiac drugs. In certain embodiments, the method will yield more accurate functional, genetic and epigenetic information about the effects of cardiac drugs on cardiac cells.

### Kits

The disclosure provides kits for the treatment or prevention of a cardiac conditioned mentioned above. In certain embodiments, the kit includes a therapeutic composition containing an effective amount of a peptide amphiphile disclosed herein and optionally a cardiac inducible molecule and optionally sterilized water and/or a syringe.

In certain embodiments, the kit includes a therapeutic composition containing an effective amount of a molecular beacon and a peptide amphiphile disclosed herein and optionally a cardiac inducible molecule and optionally sterilized water and/or a syringe. In certain embodiments, the molecular beacon targets human myosin heavy chain 6/7 mRNA or CD31. In certain embodiments, the cardiac inducible molecule is used to transform cells, e.g., isolated cells, stem cells, pluripotent stem cells, into cardiac cells. In certain embodiments, the molecular beacon is used to isolate a specific population of cardiac cells or cardiac precursor cells. In certain embodiments, the molecular beacon and optionally the cardiac inducible molecule are used to produce a population of cardiac cells which are combined with a peptide amphiphile for transplantation into a patient in need thereof. In certain embodiments, the kit is used to produce a therapeutic for treatment or prevention of a cardiac condition.

In certain embodiments, the kit includes a therapeutic composition containing an effective amount of a CD31 binding moiety and a peptide amphiphile disclosed herein and optionally a cardiac inducible molecule and optionally sterilized water and/or a syringe. In certain embodiments, the CD31 binding moiety is fluorescent. In certain embodiments, the CD31 binding moiety is an antibody. In certain embodiments, the CD31 antibody is fluorescent. In certain embodiments, the kit further comprises a secondary fluorescent antibody that binds the CD31 binding moiety. In certain embodiments the CD31 binding moiety is used to isolate a specific population of cardiac precursor cells from a pool of adult stem cells (such as bone marrow cells or peripheral blood). In certain embodiments, the cardiac inducible molecule is used to transform adult stem cells into cardiac cells or cardiomyocytes. In certain embodiments, the CD31 binding moiety and optionally the cardiac inducible molecule are used to produce a population of cardiac cells which are combined with a peptide amphiphile for transplantation into a patient in need thereof. In certain embodiments, the kit is used to produce a therapeutic for treatment or prevention of a cardiac condition.

Cardiac or cardiac precursor cells isolated from a subject may be manipulated as disclosed herein and mixed with the peptide amphiphiles and water then further administered to a subject via the syringe. In some embodiments, the kit comprises a sterile container which contains a therapeutic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired, the therapeutic composition is provided together with instructions for administering it to a subject having or at risk of developing a cardiac condition. The instructions will generally include information about the use of the composition for the treatment or prevention of a cardiac condition to a patient in need thereof. In other embodiments, the instructions include at least one of the following: description of the composition, dosage schedule and administration for treatment or prevention of a cardiac condition or symptoms thereof precautions; warnings; indications; counter-indications; overdosage information; adverse reactions; animal pharmacology; clinical studies; and/or references. The instructions may be printed directly on the container (when present), or as a label applied to the container or as a separate sheet, pamphlet, card or folder supplied in or with the container.

### EXAMPLES

The following examples are put forth so as to provide those of ordinary skill in the art with a complete disclosure and description of how to make and use the assay, screening, and therapeutic methods of the disclosure, and are not intended to limit the scope of what the claimed embodiments.

### Peptide Amphiphile Synthesis

Two peptide amphiphiles, C₁₆-GTAGLIGQRGDS (PA-RGDS) and C₁₆-GTAGLIGQS (PA-S), were synthesized via Fmoc-chemistry using an Aapptech™ Apex 396 peptide synthesizer. The peptides were then alkylated at the N-termini via two 12 h reactions with palmitic acid and a mixture of o-benzotriazole-N,N,N′,N′-tetramethyluronium hexafluorophosphate and diisopropylethylamine dissolved in dimethylformamide. This was succeeded by cleavage from the resin and deprotection for 3 h, using a 40:1:1:1 cocktail of trifluoroacetic acid (TFA), deionized water, triisopropylsilane, and anisole. The collected samples were subjected to rotary evaporation to remove excess TFA, precipitated in ether, and lyophilized. Matrix-assisted laser desorption ionization time-of-flight (MALDI-TOF) mass spectrometry was utilized to confirm successful synthesis of PAs.

### Self-Assembly of Peptide Amphiphile Gels

Stock solutions of PA-RGDS and PA-S [2% (weight/volume)] were individually prepared, and their respective pH was adjusted to 7 using sodium hydroxide (NaOH). The two PAs were then mixed in 1:1 molar ratio, and self-assembly into three-dimensional hydrogels was induced by combining 50 μL of PA solution with a mixture containing 15 μL of 0.1 M CaCl₂ and 25 μL of cell suspension. The molar ratio of PA and Ca2+ was held constant at 2.

Enrichment of mESC-Derived Cardiomyocytes Using Lactate

To enrich the CMs from mESC differentiation cultures, lactate-based purification was applied. The cardiomyogenically differentiated mESCs at day 11 were dissociated with trypsin (0.05%), replated in fibronectin-coated dishes containing freshly prepared glucose-free advanced DMEM (no glucose, no pyruvate; Invitrogen) supplemented with 1 mM lactate, 0% FBS, 1% nonessential amino acids, 1% 1-glutamine, 1% (3-mercaptoethanol, and 1% penicillin/streptomycin, and continuously cultured for 7 days. Medium was changed every 2 days in order to eliminate dead cells. After 7 days of culture in lactase-containing media, the enriched mESC-derived CMs were dissociated with trypsin (0.05%), transferred to fibronectin-coated dishes, and further cultured with α-MEM supplemented with 3% FBS and ITS. To quantify the CMs, flow cytometric analysis following cell permeabilization was performed using ACTN2 antibody (Sigma).

### Induction of Myocardial Infarction and Cardiomyocyte Transplantation

Myocardial infarction (MI) was induced in mice by ligation of the left anterior descending coronary artery, and cells or other reagents were intramyocardially injected through 30 G needles at two different sites in the border zone of the myocardium immediately after surgery. Mice were randomly assigned into four treatment groups: (i) PBS as a control group, (ii) 2×10⁵ mESCs-derived CMs only, (iii) PA-RGDS only, or (iv) 2×10⁵ mESCs-derived CMs encapsulated with PA-RGDS. Due to the limiting factor of murine heart size and the requirement to deliver a large quantity of cells, only one concentration ratio of PA (2×10⁵ mESCs-derived CMs) to cells was used for the studies. All CMs were obtained from the same differentiation batch and prelabeled with CM-DiI (red fluorescence) before cell injection for cell tracking in histology.

Assessment of Cardiomyocyte Stability After Encapsulation with PA-RGDS

To examine the compatibility of the PA-RGDS as a scaffold to encapsulate CMs and the cellular response of CMS against PA-RGDS, live/dead staining was performed against CM/PA-RGDS constructs which were harvested after 7 days of cultivation in normal CM culture condition. A majority of RNVCMs encapsulated with PA-RGDS were alive even after 7 days of culture and very few dead cells were present indicating that PA-RGDS minimally affected the viability of the CMs over 7-day culture period (Live: 90.7±6.2% vs. Dead: 9.3±0.8%, respectively, FIG. 2).

### Investigation of Cytoprotective Effects of PA-RGDS Encapsulation Against Simulating Ischemic Injury Model In Vitro

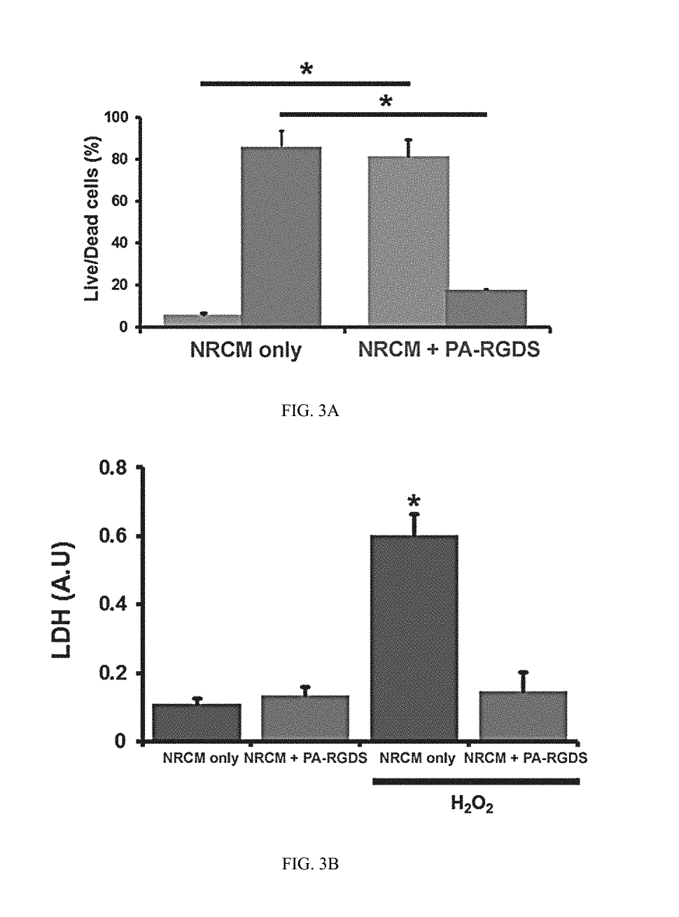

Since oxidative stress in the ischemic heart is increasingly recognized as a possible cause for poor survival of transplanted cells, whether encapsulation of the CMs with PA-RGDS increases their survival when exposed to the H₂O₂ simulating ischemic condition in vitro was investigated. The results from live/dead staining demonstrated that encapsulation of RNVCMs with PA-RGDS significantly improved CM viability as substantially higher percentage of live CMs were detected in CM/PA-RGDS constructs in compared to bare CMs control group (Live: CM/PA-RGDS: 81.7±9.3% vs.CM only: 6.1±0.4%; Dead: CM/PA-RGDS: 17.7±2.3% vs.CM only: 86.1±7.7%; FIG. 3A). Similarly, CM/PA-RGDS constructs exposed to H₂O₂ released a significantly lower level of LDH in comparison to CM only group (CM/PA-RGDS: 0.14±0.05 arbitrary units (AUs) vs. CM only: 0.61±0.07 AU; FIG. 3B), indicating that encapsulating the CMs with PA-RGDS is capable of improving CM survival against H202-induced oxidative stress.

### Increased Retention of Transplanted Cardiomyocyte Through PA-RGDS

To investigate the effects of PA-RGDS on CM engraftment and survival in vivo, cultured HL-1 CMs were pre-labeled with CM-DiI, a red fluorescent dye, and injected into the hearts of athymic nude mice (2×10⁵ cells per mouse) in the absence or presence of PA-RGDS. For more accurate quantification of these engrafted/survived cells, flow cytometry analysis was performed with enzymatically digested heart tissues after 7 days of injection (FIG. 4). Approximately 4% of DiI positive HL-1 CMs were detected in the mouse heart tissues by flow cytometry after 7 days of injection. Of interest, injection of HL-1 CMs with PA-RGDS significantly improved (˜3 folds) the rate of engraftment/survival of HL-1 CMs (CM/PA-RGDS: 11.7±1.2% vs. CM only: 4.4±0.5%). Furthermore, the results from immunohistochemistry with Actn2 (or α-sarcomeric actinin) antibody revealed that the significant numbers of DiI positive CMs expressing cardiac specific protein Actn2 from the heart tissues were observed 7 days after injection of CMs when they were injected with PA-RGDS. The results from both flow cytometry and immunohistochemistry strongly indicated that implementation of PA-RGDS greatly increased the retention rate of the injected CMs into the hearts in vivo.

Generation and Purification of Cardiomyocytes from Mouse Embryonic Stem Cells

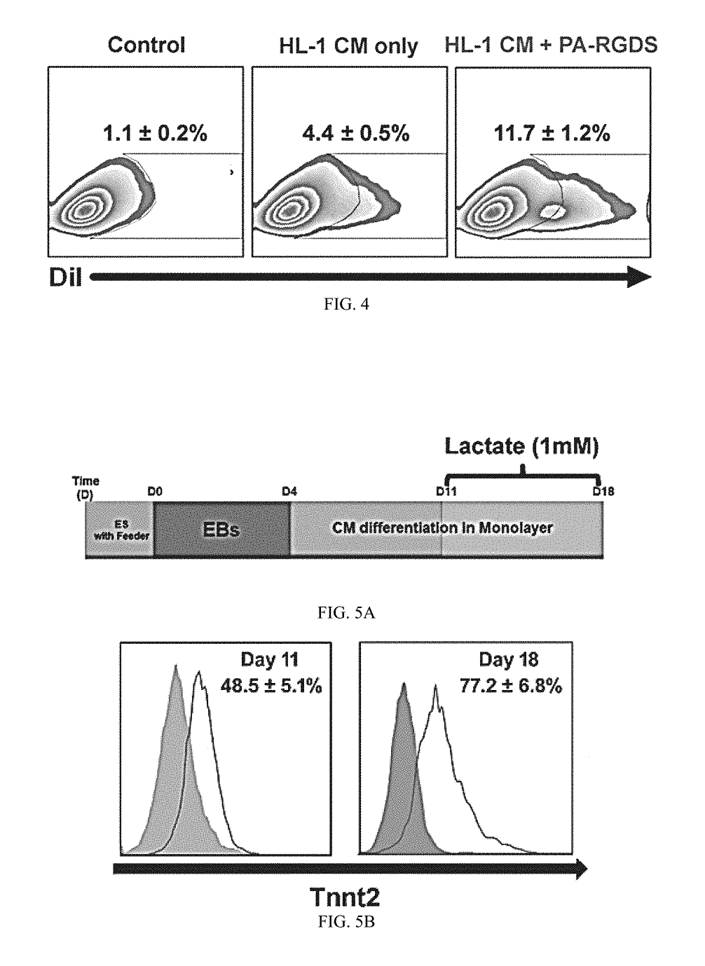

CMs were generated from mESCs by establishing an embryoid body (EB)-mediated CM differentiation system (FIG. 5A). Undifferentiated mouse ESCs (J1) maintained on STO feeder cells were enzymatically detached to form EBs. Subsequently, day 4 EBs were plated into a fibronectin-coated dish for further CM differentiation. Spontaneously beating clumps began to appear 3-4 days after the plating of EBs on the monolayer cultures in the presence of ascorbic acid (50 μg/m1). After 7 days of CM differentiation process, the culture condition was modified to glucose-depleted media supplemented with 1mM lactate in order to enrich mESC-derived CMs. This lactate-based CM purification was continued for 7 days. To further characterize these enriched mESC-derived CMs through lactate based purification method, we carried out both intracellular flow cytometry analysis using Tnnt2 antibody and immunocytochemistry. The results from intracellular flow cytometry analysis showed that the percentage of Tnnt2 positive cells were 79.9±4.5% (FIG. 5B). In addition, the immunocytochemistry results demonstrated that almost all of CMs-derived mESCs highly expressed CM specific proteins such as Actn2, Tnnt2, and Myh6/7 confirming their CM nature. Collectively, these results indicated that CMs were successfully generated and enriched through our CM differentiation and purification system.

### CM/PA-RGDS Improved Cardiac Function and Reduced Scar Formation After MI

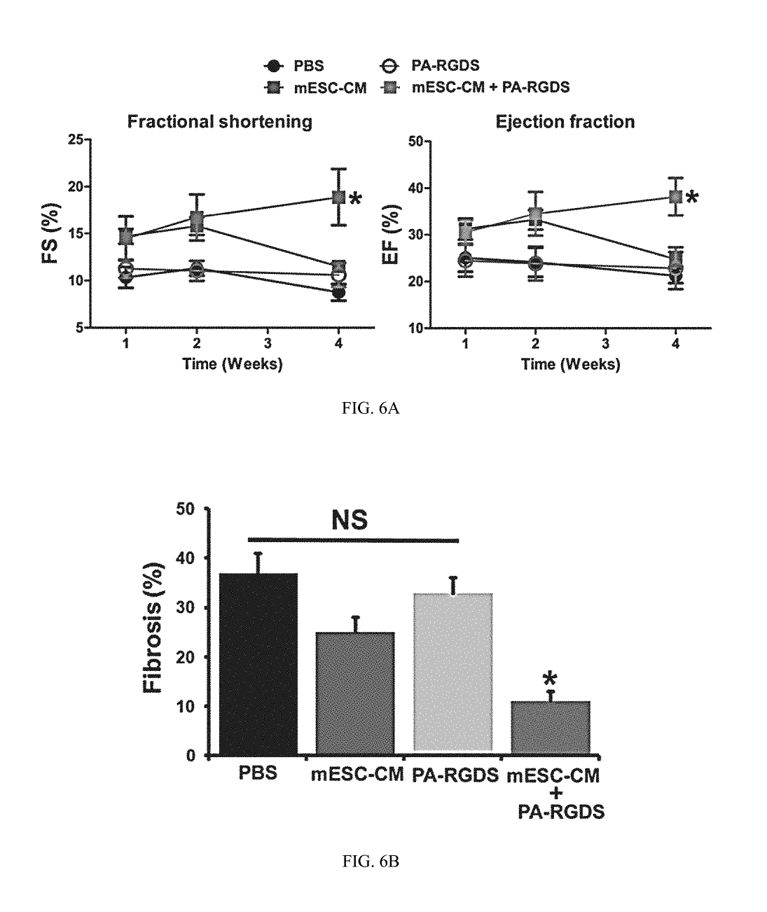

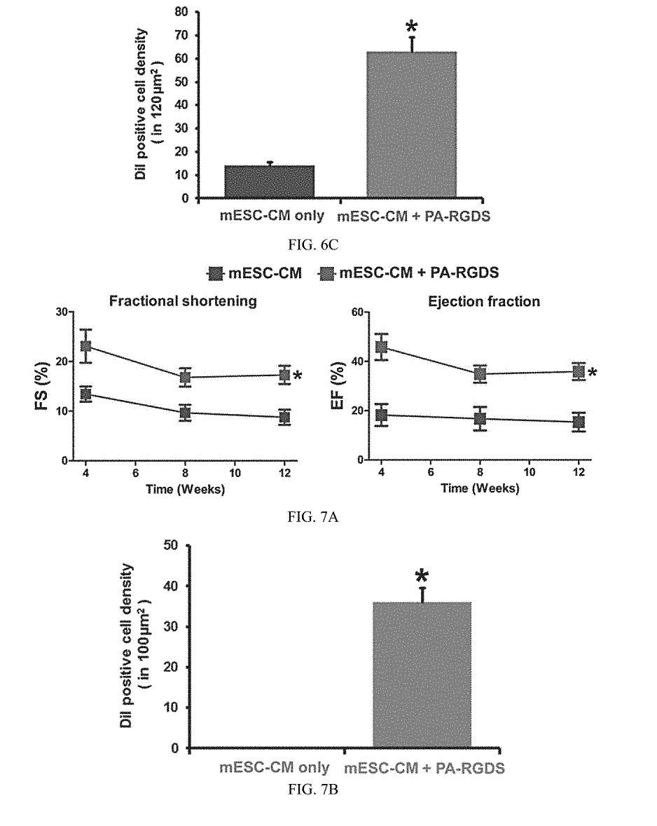

Next, the therapeutic effects of treatments with CM only, PA-RGDS only and mESC-CMs with PA-RGDS, comparing with PBS control in ischemic myocardium in mice was investigated. Echocardiography was performed regularly to measure cardiac remodeling and function (FIG. 6A). One week post MI induction and treatment echocardiographic assessments of ejection fraction (EF) and fractional shortening (FS) did not differ significantly among the treatment groups. However, although differences did not reach statistical significance, both EF and FS of the two experimental groups (CM and CM+PA-RGDS) were higher than those of the PA-RGDS only and PBS group from week 2. Of note, while the cardiac function in CM or PA-RGDS only groups was dramatically reduced between 3-4 weeks, cardiac function of the CM+PA-RGDS group was preserved and significantly greater than the cardiac functions of the three other groups until 4 weeks after MI induction (FIG. 6A).

Masson's Trichrome staining was performed with fixed sections of infarcted hearts to evaluate the extent of scar formation 4 weeks after MI induction. Quantification of the scar areas showed that the treatment with encapsulated mESC-CM with PA-RGDS significantly reduced scar area (14.7±2.1%; N=6) compared to the treatment with other three experimental groups (PBS control: 44.2±5.1%; CM only: 32.4±2.6%; and PA-RGDS only: 41.7±4.7%; N=6) (FIG. 6B).

Histological analyses exhibited that many clusters of DiI labelled CMs were identified within the peri-infarct and infarct regions in the heart tissues that received CMs with PA-RGDS after 4 weeks post treatment. In contrast, heart tissues that received CMs only had fewer numbers of DiI labelled CMs detected, CMs injected with PA-RGDS has substantially more number of DiI positive cells were observed (FIG. 6C). Furthermore, immunohistochemistry images of heart tissues harvested after 4 weeks post MI induction and treatment showed that intramyocardially injected CMs with PA-RGDS engrafted robustly in the myocardium, formed clusters, integrated into the host myocardium, and expressed representative CM proteins such as Tnnt2 and MHC6/7, indicating that the treatment of CMs with PA-RGDS markedly enhance cell retention and engraftment within the damaged myocardium.

Sustained Therapeutic Effects of mESC-CMs with PA-RGDS

Prior studies reported that the beneficial effects from treatment with various types of stem cells or stem cell derived cells were substantially diminished by 3-4 weeks, mostly due to massive cell death or ejection from the myocardium. Hence, the long-term therapeutic effects of CM/PA-RGDS treatment was continuously monitor in comparison with those of CM only injection group. Echocardiography data from a subset of mice that received CMs with PA-RGDS had sustained their improved cardiac function until week 12 compared with the data from the CM only injected group that showed progressive and marked decline in cardiac function. (FIG. 7A)

Of note, substantial numbers of DiI positive CMs were still able to be identified in the heart tissues receiving CMs with PA-RGDS even after 14 weeks (FIG. 7B). In contrast, the heart tissues receiving CMs had only little number of DiI labeled CMs identified. Moreover, immunohistochemistry images of heart tissues harvested after 14 weeks post MI induction and treatment showed that intramyocardially injected CMs with PA-RGDS were survived well and structurally integrated into the host myocardium as determined by expressions of CM representative proteins such as Tnnt2 and MHC6/7, indicating that the treatment of CMs with PA-RGDS markedly enhance cell retention and engraftment within the damaged myocardium.

1. A composition comprising
a) a peptide amphiphile comprising a cell adhesive peptide sequence and a protease degradable sequence linked to a hydrocarbon,
wherein the cell adhesive peptide sequence and protease degradable sequence is GTAGLIGQRGDS (SEQ ID NO: 1); and
b) a cardiac cell or a cardiac precursor cell,
wherein the cardiac cell or cardiac precursor cell is made by the process of culturing a stem cell and ascorbic acid under conditions such that cell replication results in cells that that increase expression of a cardiac-specific nucleic acid selected from Nkx2.5, α-actin, MLC-2v, and MLC-2a.

2. The composition of claim 1, wherein cell replication results in cells that spontaneously contract.

3. The composition of claim 1, wherein the stem cell is an embryonic stem (ES) cell or embryoid body cell.

4. The composition of claim 1, wherein the cardiac cell or cardiac precursor cell is isolated from a sample by fluorescence-activated cell sorting after mixing with a molecular beacon targeting a cardiac-specific nucleic acid.

5. The composition of claim 1, wherein the stem cell expresses CD31 and is isolated from bone marrow or peripheral blood by selecting cells that express CD31.

6. The composition of claim 1, wherein the peptide amphiphile is CH₃(CH₂)₁₄CONH-GTAGLIGQ-RGDS (SEQ ID NO: 1).

7. A method of treating or preventing cardiac condition comprising administering a composition comprising cultured cardiac cells or cardiac precursor cells and a peptide amphiphile of claim 1 in an effective amount to a subject in need thereof.

8. The method of claim 7, wherein the cardiac condition is peripheral vascular disease, myocardial ischemia, heart failure, or stroke.

9. The method of claim 7, wherein the subject is a human.


SEQUENCE LISTING (20 sequences)

SEQ ID NO: 1 - protein, 12 aa, Artificial
  Synthetic construct
GTAGLIGQRGDS

SEQ ID NO: 2 - protein, 8 aa, Artificial
  Synthetic construct
GTAGLIGQ

SEQ ID NO: 3 - protein, 4 aa, Artificial
  Synthetic construct
DGEA

SEQ ID NO: 4 - protein, 5 aa, Artificial
  Synthetic construct
YIGSR

SEQ ID NO: 5 - protein, 5 aa, Artificial
  Synthetic construct
IKVAV

SEQ ID NO: 6 - protein, 5 aa, Artificial
  Synthetic construct
ERGDS

SEQ ID NO: 7 - protein, 6 aa, Artificial
  Synthetic construct
KQAGDV

SEQ ID NO: 8 - protein, 3 aa, Artificial
  Synthetic construct
LDV

SEQ ID NO: 9 - protein, 3 aa, Artificial
  Synthetic construct
IDS

SEQ ID NO: 10 - protein, 3 aa, Artificial
  Synthetic construct
RLD

SEQ ID NO: 11 - protein, 5 aa, Artificial
  Synthetic construct
KRLDS

SEQ ID NO: 12 - protein, 3 aa, Artificial
  Synthetic construct
LET

SEQ ID NO: 13 - protein, 3 aa, Artificial
  Synthetic construct
IET

SEQ ID NO: 14 - protein, 6 aa, Artificial
  Synthetic construct
YYGDLR

SEQ ID NO: 15 - protein, 6 aa, Artificial
  Synthetic construct
FYFDLR

SEQ ID NO: 16 - protein, 3 aa, Artificial
  Synthetic construct
RGD

SEQ ID NO: 17 - protein, 9 aa, Artificial
  Synthetic construct
GTAGLIGQS

SEQ ID NO: 18 - protein, 11 aa, Artificial
  Synthetic construct
GTAGLIGKKKK

SEQ ID NO: 19 - protein, 12 aa, Artificial
  Synthetic construct
GTAGLIGKKKKK

SEQ ID NO: 20 - protein, 13 aa, Artificial
  Synthetic construct
GTAGLIGKKKKKK